(19) United States
(12) Patent Application Publication
(10) Pub. No.: US 2005/0267553 A1
(43) Pub. Date: Dec. 1, 2005

(54) System and method for controlling electrical stimulation and radiofrequency output for use in an electrosurgical procedure

(76) Inventors: Doug Staunton, Kalamazoo, MI (US); Karen Staley, Kalamazoo, MI (US)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 11/122,702

(22) Filed: May 5, 2005

Related U.S. Application Data
(60) Provisional application No. 60/568,186, filed on May 5, 2004.

(51) Int. Cl. A61N 1/18; A61F 2/00

(52) U.S. Cl. 607/101; 607/3; 607/48

(57) ABSTRACT

A system (20) for performing an electrosurgical procedure includes a first electrode (22) for contacting a target nerve tissue area of a patient to deliver electrical energy to the target nerve tissue area. The system (20) is characterized by a multi-function hand controller (30) in communication with a control unit (24) and remote from and corresponding to a screen unit (138) for providing inputs to the control unit (24) in parallel with the screen unit (138) whereby an operator may position the multi-function hand controller (30) at a patient's side and enter inputs to said control unit (24) by either of said multi-function hand controller (30) and the screen unit (138). The multi-function hand controller (30) includes a plurality of push-buttons (52) corresponding to the screen unit (138) for entering inputs to the control unit (24). The inputs to the control unit (24) control the electrical energy.

### CROSS REFERENCE TO RELATED APPLICATION

This application claims the benefit of application Ser. No. 60/568,186 filed May 5, 2004, the advantages and disclosure of which are hereby incorporated by reference.

### BACKGROUND OF THE INVENTION

1. Field of the Invention

The invention relates to a system for performing an electrosurgical procedure using an electric stimulator integrated with a radiofrequency generator and a method of operating such a system.

2. Description of the Prior Art

In the field of electrosurgery, it is well known to contact an electrode to a target nerve tissue area of a patient for delivery of radiofrequency output through the electrode to the target nerve tissue area. The delivery of the radiofrequency output through the electrode to the target nerve tissue area is used to cut or coagulate the target nerve tissue area or to create a lesion in the target nerve tissue area. Generally the electrode is in communication with a control unit for controlling the delivery of the radiofrequency output to the electrode. More specifically, radiofrequency output is delivered to the target nerve tissue area to create a lesion to interrupt nerve communication. Lesion creation generally includes the steps of sensory stimulation, motor stimulation, and lesion creation. Sensory stimulation is used to facilitate the proper placement of the electrode before creating the lesion. Motor stimulation is used to avoid proximity of the electrode to the motor nerve before lesion creation to prevent inadvertent damages. And lesion creation exposes the target nerve tissue area to radiofrequency output to create the lesion to interrupt a nerve path. Alternatively, radiofrequency energy may be applied with a low duty cycle to prevent creation of a lesion, but still deliver an intense electric field to the target tissue. This intense electric field influences nerve fiber transmission and can provide a more conservative treatment option to lesion creation.

During the course of the procedure, it is necessary to alternate between electrical stimulation pulses and radiofrequency output. Each of the sensory stimulation, the motor stimulation, and the lesion creation utilize different electrical outputs. In addition, the stimulation and radiofrequency specifications vary with patients and procedures. Specific examples of such specifications which require changing, among others, include amplitude, frequency, temperature, duration, and radiofrequency and on time settings.

Several electrosurgical apparatus are known in the field to include a user interface for changing the specifications of the stimulation and radiofrequency output. One particular type of user interface is a touch-sensitive screen for entering inputs to a control unit to control the delivery of the stimulator or radiofrequency output to the electrode. Such a system is shown in the U.S. Patent Application Publication 2004/0082946 to Malis et al. This system includes a touch-sensitive screen in communication with the control unit for providing inputs to the control unit. The operator is able to change the specifications of the stimulation or radiofrequency output by touching touch-buttons on the touch-sensitive screen. After changing the output specifications, the operator may touch touch-buttons on the touch-sensitive screen to deliver output to the target nerve tissue area. However, this system requires the operator to be located next to the touch-sensitive screen to change the specifications of the output.

Some current systems provide a foot switch, which is limited in function to turning output power on and off and are of very limited practical use since an operator is still required to make setting adjustments on a control console. Due to the ergonomic issues and difficulty attaining direct sight of footswitches, they do not lend themselves to multi-function control of the complex user interfaces with potentially dangerous outputs.

### SUMMARY OF THE INVENTION AND ADVANTAGES

The invention is characterized by a multi-function hand controller positioned at the side of the patient and remote from the touch-sensitive screen and for operating the multi-function hand controller corresponding to the touch-sensitive screen for entering inputs to the control unit in parallel with inputs to the touch sensitive screen. The inputs to the control unit may be made by either of the multi-function hand controller at the patient's side and the touch-sensitive screen remote from the patient. The inputs to the control unit control the delivery of the stimulation or radiofrequency output, which is delivered to a target nerve tissue area of a patient through an electrode.

The invention also includes a method characterized by the steps of positioning the multi-function hand controller at the side of the patient and remote from the touch-sensitive screen and operating the multi-function hand controller corresponding to the touch-sensitive screen.

The current systems and methods do not include a multi-function hand controller corresponding to a touch sensitive screen for entering inputs to the control unit in parallel with inputs to the touch-sensitive screen. Therefore, the current systems require the operator to remain near the touch-sensitive screen to enter inputs to the control unit.

Accordingly, because the multi-function hand controller is positioned at the patient's side, the operator is not restricted to remain near the touch-sensitive screen but may be positioned at the patient's side and enter inputs to the control unit with the multi-function hand controller.

Some of the current systems provide a foot switch, which is limited in function to turning output power on and off and is of very limited practical use since an operator is still required to make setting adjustments on a control console. Due to ergonomic issues and difficulty attaining direct sight of footswitches, the foot switches do not lend themselves to multi-function control of complex user interfaces with potentially dangerous outputs. A multi-function hand controller avoids these problems by giving control of multiple functions in an easy to see and manipulate device.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 is a perspective view of a system for generating radiofrequency output for use in an electrosurgical procedure;

FIG. 2 is a general schematic block diagram of a system for generating radiofrequency output for use in an electrosurgical procedure;

FIGS. 3-14 is a screen diagram for a touch-sensitive screen of the system from FIG. 1;

FIG. 15 is a perspective view of an alternative embodiment of a multi-functional hand controller; and

FIG. 16-18 is a perspective view of a protective bag for a multi-functional hand controller.

### DETAILED DESCRIPTION OF THE INVENTION

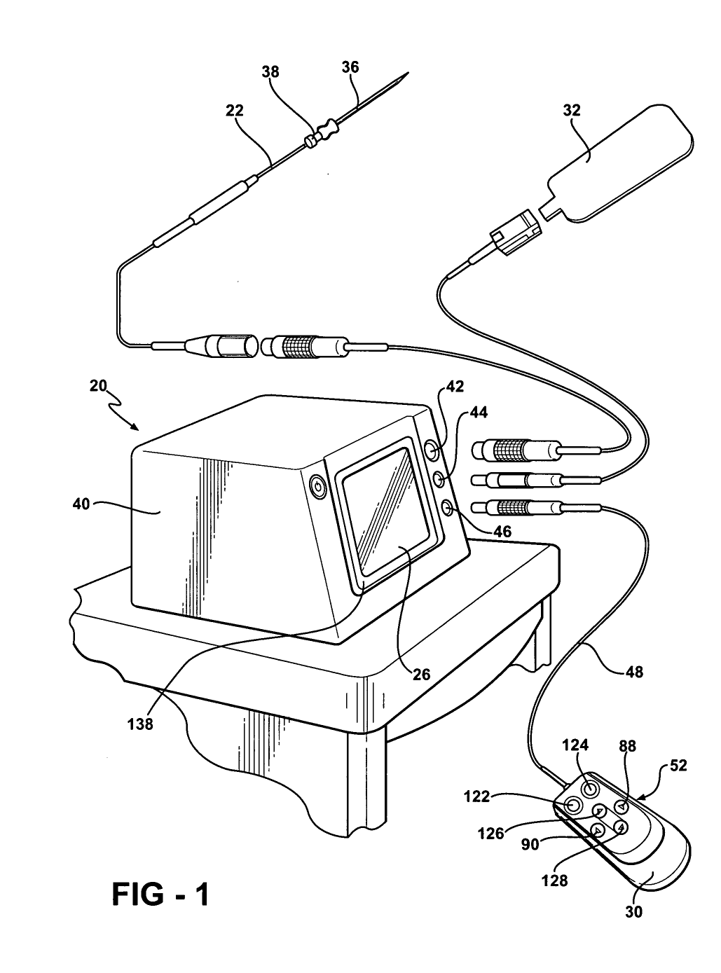

Referring to the Figures, wherein like numerals indicate like or corresponding parts throughout the several views, a system for generating radiofrequency output for use in electrosurgical procedures is shown generally at 20 at in FIG. 1. The subject invention may be employed with several systems, including the system disclosed in the United States Patent Application Publication 2004/0082946 to Malis et al, which is hereby incorporated by reference.

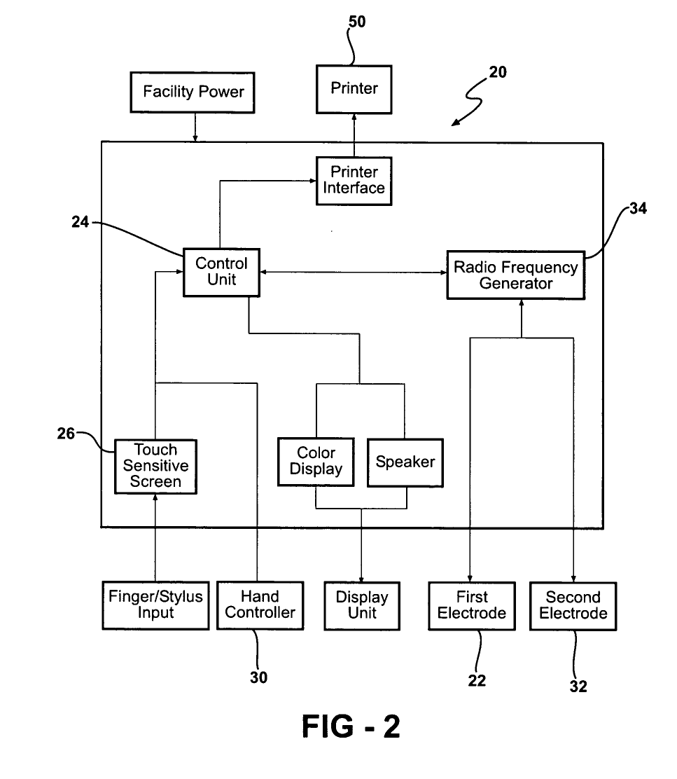

Referring to FIGS. 1 and 2, the system 20, for generating electrical energy for use in an electrosurgical procedure, includes a first electrode 22 for contacting a target nerve tissue area of a patient and for delivering the electrical energy to the target nerve tissue area. The electrical energy includes stimulation energy for performing stimulation to assure proper placement of the first electrode as well as radiofrequency energy for creation of a lesion.

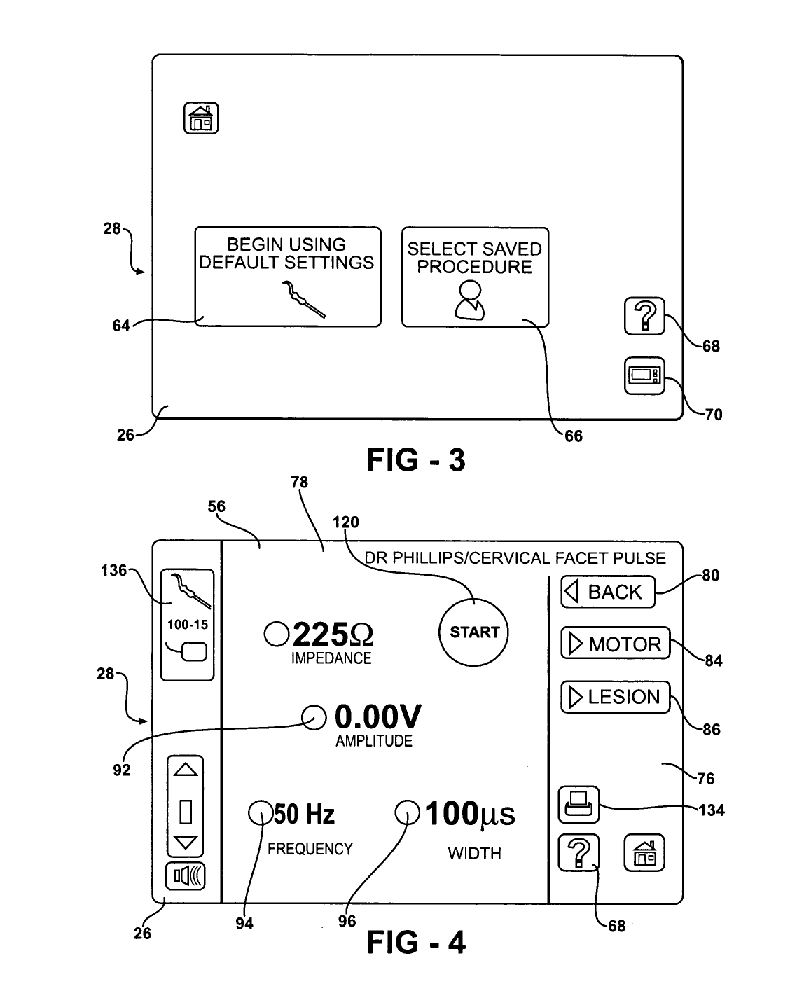

The system 20 further includes a control unit 24 for controlling the delivery of the electrical energy to the first electrode 22 and a screen unit 138 for displaying a plurality of screen views 28 and in communication with the control unit 24 for navigating through the plurality of screen views and for providing inputs to the control unit 24 for controlling the delivery of electrical energy to the first electrode 22. In the preferred embodiment the screen unit 138 includes a touch sensitive screen 26 responsive to touching for navigating through the plurality of screen views 28, as shown in FIGS. 3-14, and for providing inputs to the control unit for controlling the delivery of electrical energy to the first electrode 22. The touch-sensitive screen 26 is of the type well known in the art and responds to the touch of a finger or a stylus. The touch-sensitive screen 26 presents a plurality of touch-buttons responsive to touching for navigating through the plurality of screen views 28 and for providing inputs to the control unit 24. Alternatively, the system cost could be reduced by eliminating the touch-sensitive screen 26 and placing buttons on the margin of the screen unit 138 which would function in accordance with adjacent on-screen labels.

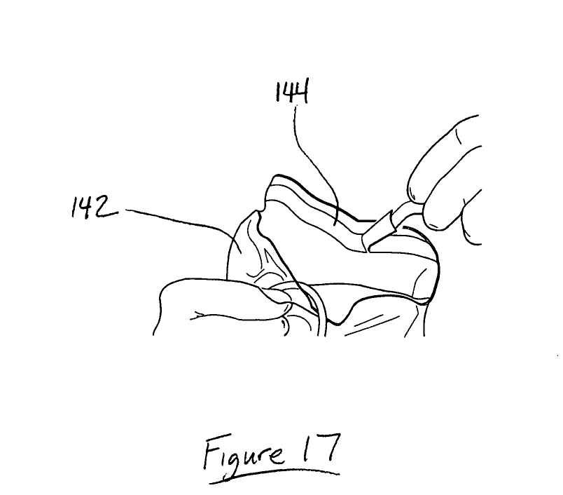

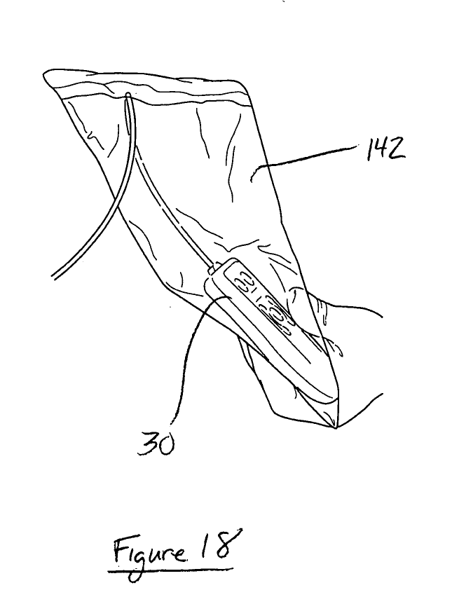

The system 20 is characterized by a multi-function hand controller 30 in communication with the control unit 24 and remote from the screen unit 138 for providing inputs to the control unit 24. An operator may position the multi-function hand controller 30 at the patient's side and enter inputs to the control unit 24 by either of the multi-function hand controller 30 and the screen unit 138. In addition, the multi-function hand controller 30 corresponds to the screen unit 138 for entering inputs in parallel to the control unit 24. In other words the operator may be located at the patient's side and not in a line of sight with the screen unit 138 while providing inputs to the control unit 24 with the multi-function hand controller 30 to perform the electrosurgical procedure. Because the multi-function hand controller 30 operates in parallel with the screen unit 138, the operator may enter some inputs to the control unit 24 through the screen unit 138 and enter other inputs to the control unit 24 through the multi-function hand controller 30. The control unit 24 includes software and inputs to the control unit 24 through either of the screen unit 138 and the multi-function hand controller 30 controls the software, as will be discussed further below. In addition, as shown in FIGS. 16-18 the multi-function hand control 30 may be sealed in a protective bag 142 by placing the multi-function hand control 30 in the bag and exposing an adhesive strip 144.

A second electrode 32 is in contact with the patient to complete the electrical circuit. In the embodiment shown in FIG. 1, the second electrode 32 is a pad for contacting the patient's skin. Alternatively, the second electrode 32 may be in the form of an electrode similar to the first electrode 22. A radiofrequency generator 34 is in communication with the first electrode 22 and is controlled by the control unit 24 for providing the stimulation and radiofrequency output to the first electrode 22. The control unit 24 is in communication with the radiofrequency generator 34 for controlling the radiofrequency generator 34. The second electrode 32 is in communication with the radiofrequency generator 34 and thus completes the electrical circuit from the radiofrequency generator 34, through the first electrode 22, through the patient, and returning through the second electrode 32 to the radiofrequency generator 34. The first electrode 22 and the second electrode 32 (as shown in the embodiment of FIG. 1) may be of the type well known in the art for performing monopolar electrosurgery, however, the subject invention is also applicable for bipolar electrodes for performing bipolar electrosurgery as well as other electrosurgical instruments for performing other electrosurgical procedures.

Referring back to FIG. 1, the system 20 also includes a cannula 36 for providing access for the first electrode 22 to the target nerve tissue area. A stylet 38 is coaxially insertable into and removable from the cannula 36 for providing structural rigidity for insertion of the cannula 36 into the target nerve tissue area and for removal of the stylet 38 after insertion of the cannula 36 into the target nerve tissue area. The first electrode 22 is in communication with the radiofrequency generator 34 for insertion into the cannula 36 after removal of the stylet 38 to contact the target nerve tissue area for delivering the electrical energy to the target nerve tissue area.

The control unit 24, the radiofrequency generator 34, and the screen unit 138 are encased in a housing 40 with the screen unit 138 mounted on a front side of the housing 40. Three electrical jacks are mounted on the front side of the housing 40. A first jack 42 and a second jack 44 are connected to the radiofrequency generator 34 and a third jack 46 is connected to the control unit 24. The first electrode 22 includes a plug for connection to the first jack 42 and the second electrode 32 includes a plug for connection to the second jack 44 thereby establishing communication between the radiofrequency generator 34 and the electrodes 22,32. The multi-function hand controller 30 includes a cord 48 attaching the control unit 24 to the multi-function hand controller 30 to establish communication between the multi-function hand controller 30 and the control unit 24. Specifically, the cord 48 includes a plug for connection of the multi-function hand controller 30 to the third jack 46. Alternatively, the multi-function hand controller 30 and the control unit 24 include a wireless communication system for establishing wireless communication between the multi-function hand controller 30 and the control unit 24. In such an embodiment, the multi-function hand controller 30 establishes communication with the control unit 24 via transmission means including radiofrequency, infrared, or ultrasound. Alternatively, the wireless communication system includes an adapter in wired communication with the control unit 24 for receiving wireless signals from the multi-function hand controller 30 and for converting the wireless signals into wired signals for communication to the control unit 24. The adapter may be plugged into the third jack 46.

The screen unit includes a touch-sensitive screen 26 responsive to touching for navigating through the plurality of screen views 28 and for providing inputs to the control unit 24 for controlling the delivery of electrical energy to the first electrode 22. The touch-sensitive screen 26 presents a plurality of touch-buttons, which will be discussed in detail below, responsive to touching for navigating through the plurality of screen views 28 and for providing inputs to the control unit 24. In addition, the multi-function hand controller 30 includes a plurality of push-buttons generally shown at 52 for entering inputs to the control unit 24. The plurality of push-buttons 52 correspond to the plurality of touch-buttons on the touch sensitive screen 26 and provide inputs to the control unit 24 in parallel with the touch-sensitive screen 26.

The touch-sensitive screen 26 presents the plurality of screen views, generally shown at 28 on FIGS. 3-14, with each screen view 28 navigable by either of the multi-function hand controller 30 and the touch-sensitive screen 26 for providing input at each of the plurality of screen views 28.

Referring to FIGS. 3-14, the plurality of screen views 28 includes a home screen view 54, a sensory stimulation screen view 56, a motor stimulation screen view 58, a lesion creation screen view 60, and a procedure summary screen view 62. More specifically, the plurality of screen views 28 corresponds to the operator's work flow during the creation of the lesion in the target nerve tissue area. Particularly, as the operator navigates through the plurality of screen views 28, the home screen view 54 is displayed first, then the sensory stimulation screen view 56 is displayed, then the motor stimulation screen view 58 is displayed, then the lesion screen view 60 is displayed, and finally the procedure summary screen view 62 is displayed. The order of the screen views corresponds with the order of the procedure as the operator will generally first perform sensory stimulation, followed by motor stimulation, followed by lesion creation. Sensory stimulation is used to facilitate the proper placement of the electrode before creating the lesion. Motor stimulation is used to avoid proximity to the motor nerve before lesion creation to prevent inadvertent damages. Lesion creation exposes the target nerve tissue area to radiofrequency output to create a lesion or high strength electric field to interrupt a nerve path.

Referring to FIG. 3, the home screen view 54 presents a plurality of touch-buttons including a default settings touch-button 64, a saved procedure touch-button 66, a help touch-button 68, and a system settings touch-button 70. The default settings touch-button 64 is touched to enter input to the control unit 24 to navigate to the sensory stimulation screen view 56 with default inputs for an electrical energy specification at each of the sensory stimulation screen view 56, motor stimulation screen view 58, and lesion creation screen view 60. The plurality of screen views 28 also includes a saved file screen view 72. The saved procedure touch-button 66 is touched to enter inputs to the control unit 24 to navigate to a saved file screen view 72 as shown in FIG. 8. The saved file screen view 72 includes saved file touch-buttons, generally shown at 74, corresponding to previously run procedures that have been saved for reuse. One of the saved file touch-buttons 74 is touched to enter inputs to the control unit 24 to navigate to the sensory stimulation screen view 56 with the electrical energy specifications for that particular saved procedure at each of the screen views 56, 58, 60. The help touch-button 68 is touched to enter inputs to the control unit 24 to navigate to a help screen view. The system settings touch-button 70 is touched to enter inputs to the control unit 24 to navigate to a system settings screen view where the operator may change system settings including default inputs for the electrical energy specifications.

The sensory stimulation screen view 56, the motor stimulation screen 58, and the lesion creation screen view 60 have a similar basic screen layout. The basic screen layout includes a menu bar 76 and an operating area 78. Each of the sensory stimulation screen view 56, the motor stimulation screen view 58, and the lesion creation screen view 60 are differently colored to aid in recognition of which screen is currently open.

The plurality of touch-buttons displayed on the menu bar 76 includes a back touch-button 80, a sensory touch-button 82, a motor touch-button 84, a lesion touch-button 86, and a summary touch-button 130. The back touch-button 80 is touched to navigate to the previous screen view. The sensory touch-button 82 is touched to navigate to the sensory stimulation screen view 56. The motor touch-button 84 is touched to navigate to the motor stimulation screen view 58. The lesion touch-button 86 is touched to navigate to the lesion creation screen view 60.

In addition, as shown in FIG. 1, the plurality of push-buttons on the multi-function hand controller 30 includes a next push-button 88 and a back push-button 90. The next push-button 88 is pressed to navigate to the next screen view in order. The back push-button 90 is pressed to navigate to the previous screen view in order. Particularly, from the sensory stimulation screen view 56, pressing the next push-button 88 will navigate to the motor stimulation screen 58. From the motor stimulation screen view 58, pressing the next push-button 88 will navigate to the lesion creation screen view 60 and pressing the back push-button 90 will navigate to the sensory stimulation screen view 56. Finally, from the lesion creation screen view 60, pressing the next push-button 88 navigates to the procedure summary screen view 62. In an alternative embodiment shown in FIG. 15, the multi-functional hand controller 30, includes only a next push-button 88 to allow space on the multi-functional hand controller 30 for other buttons to be discussed below.

The plurality of touch-buttons displayed on the operating area 78 of the sensory stimulation screen view 56 and the motor stimulation screen view 58 includes an amplitude touch-button 92, a frequency touch-button 94, and a width touch-button 96. The plurality of screen views 28 include an amplitude adjustment screen view 98, a frequency adjustment screen view 102 and a width screen view 104.

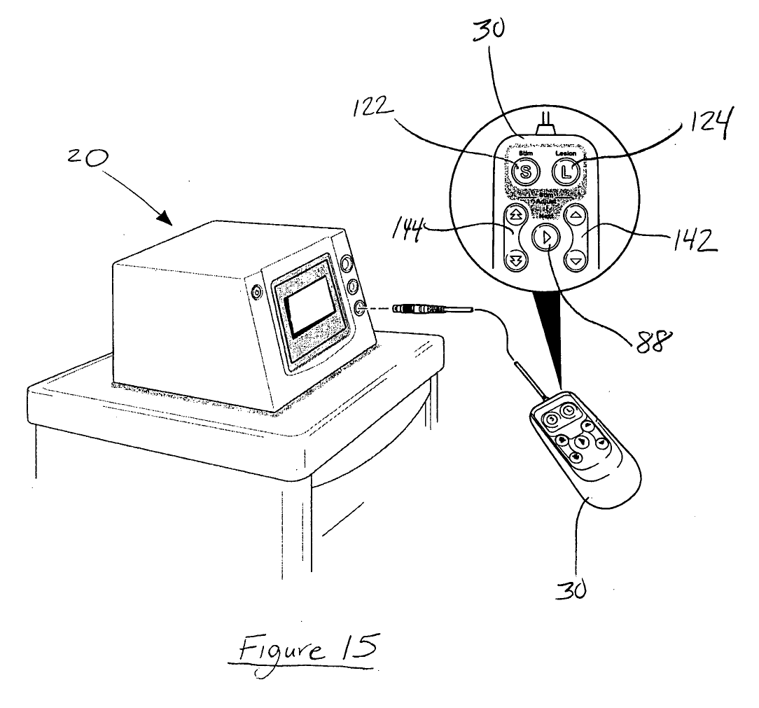

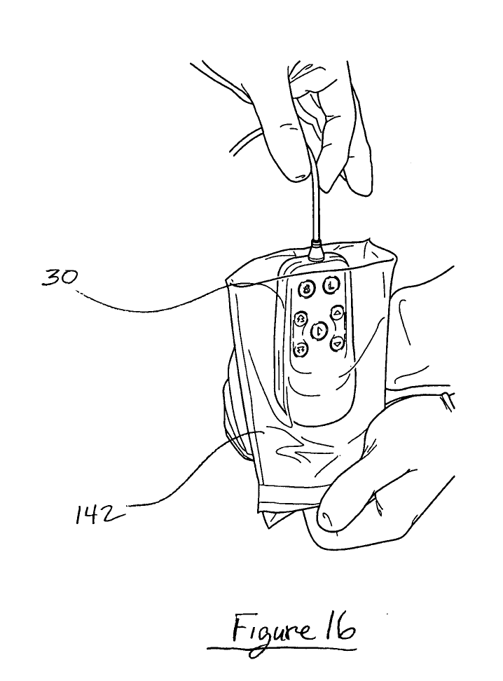

The amplitude touch-button 92 is touched to enter inputs to the control unit 24 to navigate to an amplitude adjustment screen view 98 as shown in FIG. 14 and to enter input to the control unit 24 to change an amplitude value of the stimulation energy. The plurality of touch-buttons displayed on the amplitude adjustment screen view 98 includes numbered touch-buttons generally shown at 100 for adjusting a starting amplitude value and an enter touch-button 102 to set the starting amplitude value and to return to the previous screen, where the new starting amplitude value will be displayed.

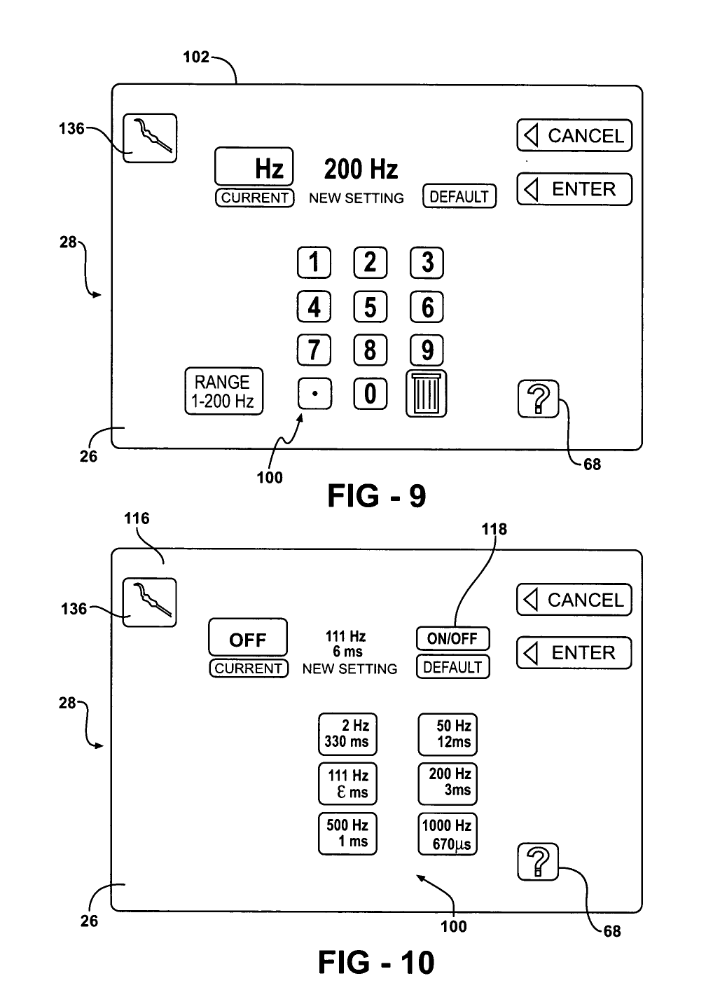

The frequency touch-button 94 is touched enter inputs to the control unit 26 to navigate to a frequency adjustment screen view 102 as shown in FIG. 9 and to enter input to the control unit 24 to change the frequency value of the stimulation energy. The plurality of touch-buttons displayed on the frequency adjustment screen view 102 includes the numbered touch-buttons generally shown at 100 for adjusting the frequency value and the enter touch-button 102 to set the frequency value and to return to the previous screen, where the new frequency value will be displayed.

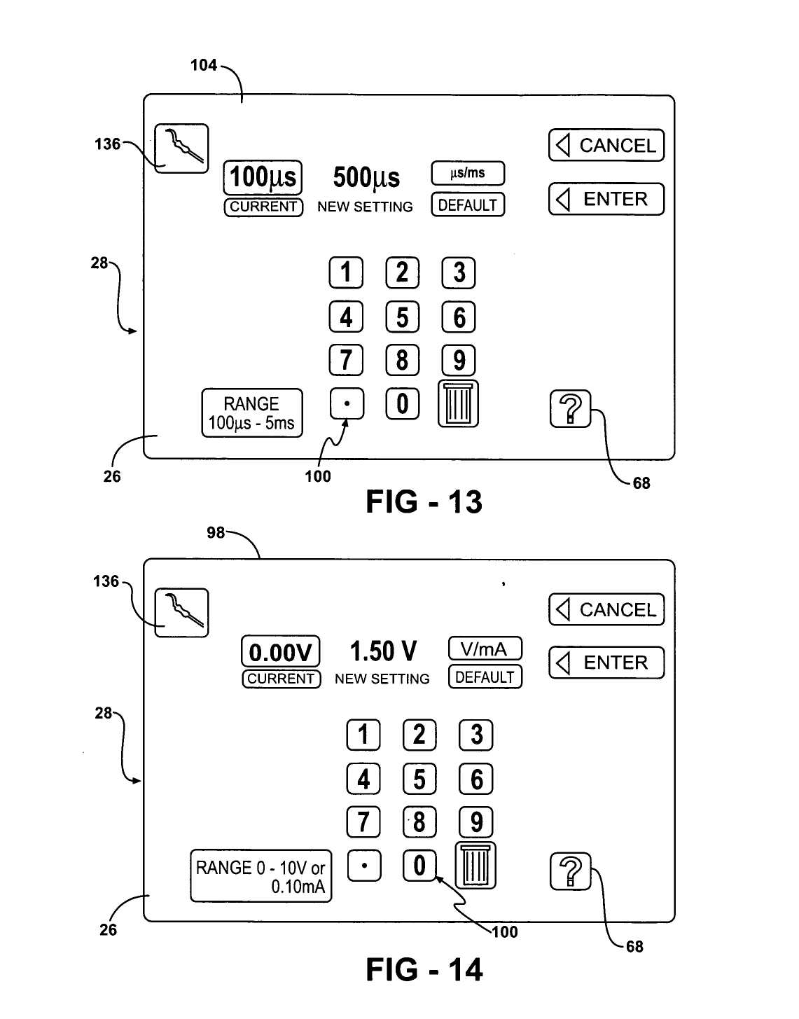

The width touch-button 96 is touched to navigate to a width adjustment screen view 104, as shown in FIG. 13, and to enter inputs to the control unit 24 to change the width value of the stimulation energy. The plurality of touch-buttons displayed on the width adjustment screen view 104 includes the numbered touch-buttons generally shown at 100 for adjusting the width value and the enter touch-button 102 to set the width value and to return to the previous screen, where the new width value will be displayed.

The plurality of touch-buttons displayed on the operating area 78 of the lesion creation screen view 60 includes a temperature limit touch-button 106, a hold time touch-button 108, and a pulse mode touch-button 110. The plurality of screen views 28 includes a temperature limit adjustment screen view 112, a hold time screen view 114, and a pulse mode adjustment screen view 116.

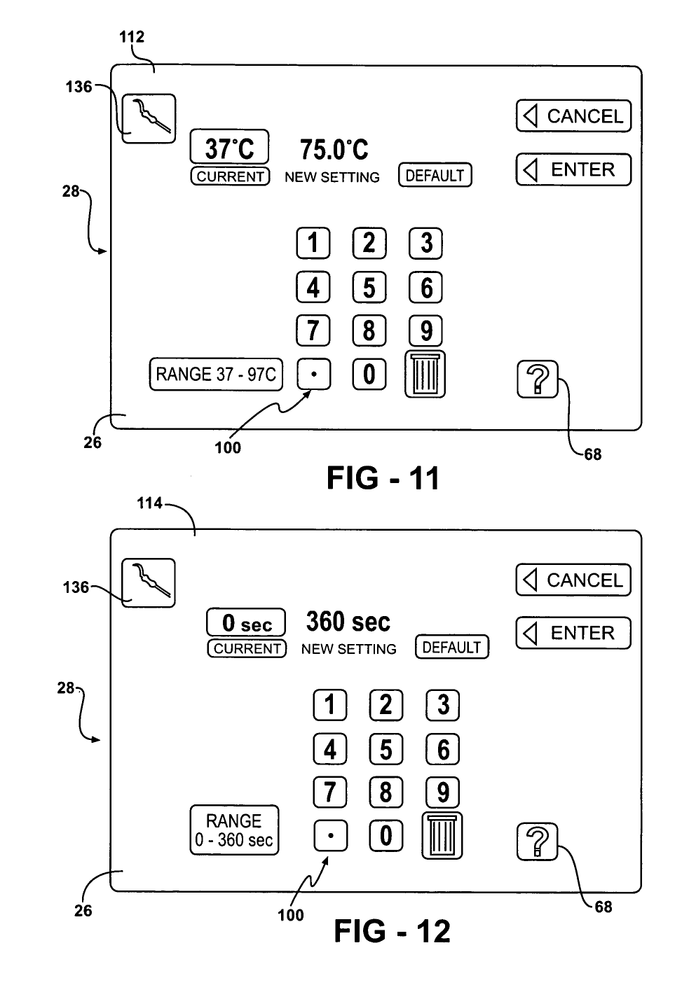

The temperature limit touch-button 106 is touched to enter input to the control unit 24 to navigate to a temperature limit adjustment screen view 112, as shown in FIG. 11, and to enter input to the control unit 24 to change a temperature limit value of the target nerve tissue area. The plurality of touch-buttons displayed on the temperature limit adjustment screen view 112 includes the numbered touch-buttons generally shown at 100 for adjusting the temperature-limit value and the enter touch-button 102 to set the temperature limit value and to return to the previous screen, where the new temperature limit value will be displayed.

The hold time touch-button 108 is touched to enter inputs to the control unit 24 to navigate to a hold time adjustment screen view 114, as shown in FIG. 12, and to enter input to the control unit 24 to change a hold time value of the radiofrequency energy. The plurality of touch-buttons displayed on the hold time adjustment screen view 114 includes the numbered touch-buttons generally shown at 100 for adjusting the hold time value and the enter touch-button 102 to set the hold time value and return to the previous screen, where the new hold time value will be displayed.

The pulse mode touch-button 110 is touched to enter inputs to the control unit 24 to navigate to a pulse mode adjustment screen view 116 as shown in FIG. 10 and to enter inputs to the control unit 24 to change a pulse mode value of the radiofrequency energy. The plurality of touch-buttons displayed on the pulse mode adjustment screen view 116 includes the numbered touch-buttons generally shown at 100 for adjusting the pulse mode value, the enter touch-button 102 to set the pulse mode value and return to the previous screen where the new pulse mode value will be displayed, and an on/off touch-button 118 to turn the pulse mode on or off.

The plurality of touch-buttons displayed on the operating area 78 also includes a start/stop touch-button 120 to enter inputs to the control unit 24 to begin or to end the delivery of the electrical to the target nerve tissue area. When electrical energy is not being delivered to the target nerve tissue area, the start/stop 120 button displays the word “start,” and toggles to display the word “stop” when the electrical energy is being delivered to the target nerve tissue area. In addition, when the start/stop touch-button 120 reads “start” the button is colored green and is circular, and when the start/stop touch-button 120 reads “stop” the button is colored, and the base screen color in the stimulation screen views 56, 58 and red in the lesion screen 60 view and is octagonal.

In addition, the plurality of push-buttons 52 on the multi-function hand controller 30 includes a stimulation push-button 122, a lesion push-button 124, an increase amplitude push-button 126, and a decrease amplitude push-button 128. The stimulation push-button 122 is pressed to begin delivery of the stimulation energy to the target nerve tissue area when the current screen view is either the sensory stimulation screen view 56 or the motor stimulation screen view 58. In addition, the increase amplitude push-button 126 is pressed during delivery of the stimulation energy to the target nerve tissue area to increase the amplitude of the stimulation energy. The decrease amplitude push-button 128 is pressed during delivery of the stimulation energy to the target nerve tissue area to decrease the amplitude of the stimulation energy. The lesion push-button 124 is pressed to begin delivery of the radiofrequency output to the target nerve tissue area when the current screen is the lesion creation screen view 58. The plurality of touch-buttons displayed on the procedure summary screen view 62 includes a record touch-button to save the inputs displayed on the procedure summary screen view 62 for reuse in a subsequent procedure, and a print touch-button 134 for printing a hard copy of the procedure summary screen view 62. In an alternative embodiment shown in FIG. 15, the multi-function hand controller includes a stimulation push-button 122 and a lesion push-button 124. In addition, the multi-function hand controller includes a fast adjustment button 140 to quickly increase and decrease the amplitude and a slow adjustment button 142 to slowly increase and decrease the amplitude.

As shown in FIGS. 4-6, the plurality of touch-buttons displayed on the screen views 56, 58, 60 includes a cannula touch-button 136 for reselecting a cannula 36. The cannula touch-button 136 is touched to change the cannula specification when a new cannula is connected to the system 20.

The printer 36 is in communication with the control unit 26 for printing a hard copy of the inputs provided to the control unit 26 and for printing the plurality of screen views 28.

The invention also includes a method of operating the system 20 for performing an electrosurgical procedure using electrical energy. The method comprises the steps of contacting the first electrode 22 to the target nerve tissue area of the patient for delivery of the electrical energy through the first electrode 22 to the target nerve tissue area. The method proceeds by manually operating the screen unit 138 to navigate through the plurality of screen views 28 and to control the delivery of electrical energy to the first electrode 22. In particular, the steps include manually operating the screen unit 138 for navigating through the plurality of screen views 28, entering inputs to the control unit 24, beginning delivery of the electrical energy to the target nerve tissue area, adjusting the inputs to the control unit, stopping the delivery of the electrical energy to the target nerve tissue area, and printing a hard copy of the inputs and the plurality of screen views 28.

The method is characterized by manually operating the hand controller 30 at the side of the patient remote from the screen unit 138 to send control signals to the control unit 24 for controlling the delivery of electrical energy to the first electrode 22. The inputs to the control unit 24 may be made by either of the multi-function hand controller 30 at the patient's side and the screen unit 138 remote from the patient. Alternatively, inputs to the control unit 24 could be entered with buttons positioned at the margin of screen unit 138 aligned with identifying graphics on screen edges, or independent functioning buttons on the screen unit 138. Because the multi-function hand controller 30 operates in parallel with the screen unit 138, the operator may enter some inputs to the control unit 24 through the screen unit 138 and enter other inputs to the control unit 24 through the multi-function hand controller 30.

The steps are further characterized by connecting the first electrode 22 to the radiofrequency generator 34 for providing the electrical energy to the first electrode 22 and connecting the second electrode 32 to the radiofrequency generator 34 and to the patient for completing the electrical circuit. By contacting the second electrode 32 to the patient and to the radiofrequency generator 34, the electrical circuit is completed from the radiofrequency generator 34, through the first electrode 22, through the patient, and returning through the second electrode 32 to the radiofrequency generator 34.

The steps are further characterized by connecting the multi-function hand controller 30 to the control unit 24 by the cord 48 for establishing communication between the multi-function hand controller 30 and the control unit 24. In addition, the method further includes connecting the printer 50 to the control unit 24 for establishing communication between the printer 50 and the control unit 24.

The steps are further characterized by inserting the cannula 36 into the target nerve tissue area for providing access for the first electrode 22 to the target nerve tissue area. The target nerve tissue area may be located in tissue deeply below the skin and even within bone and thus the cannula 36 provides access to the target nerve tissue area. The cannula 36 is subject to collapse or bending during insertion into the target nerve tissue area, thus the steps are further characterized by inserting the stylet 38 coaxially into the cannula 36 prior to insertion of the cannula 36 into the target nerve tissue area and removing the stylet 38 from the cannula 36 after insertion of the cannula 36 into the target nerve tissue area for providing structural rigidity to the cannula 36 during insertion of the cannula 36 into the target nerve tissue area. The stylet 38 also prevents coring of the tissue during insertion of the cannula 36. Removal of the stylet 38 from the cannula 36 allows the first electrode 22 to be introduced to the patient by inserting the first electrode 22 coaxially into the cannula 36 for advancing the first electrode 22 through the cannula 36 and into contact with the target nerve tissue area.

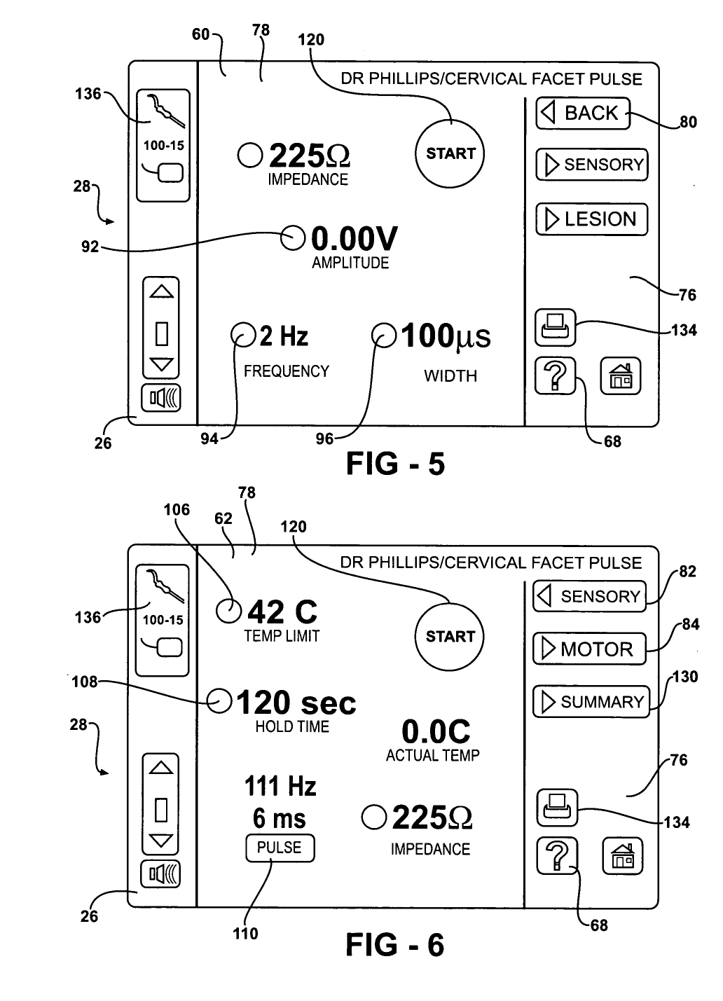

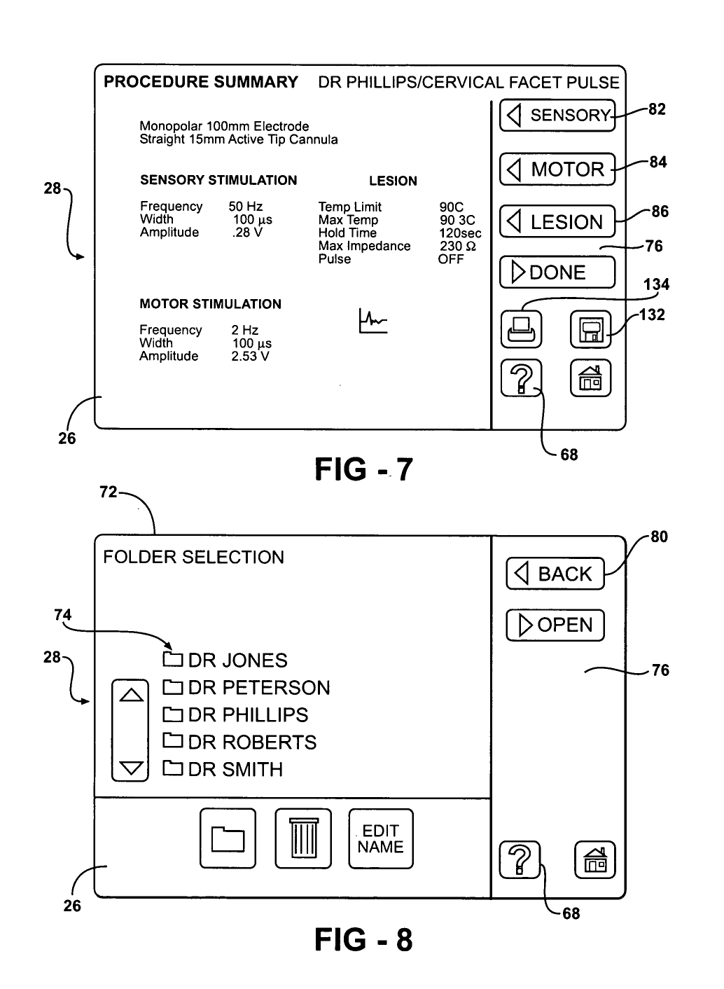

More specifically, the method is further characterized by navigating between the plurality of screen views 28 displayed on the screen unit 138 with either of the multi-function hand controller 30 and the screen unit 138 for entering inputs to the control unit 24 at one of the plurality of screen views 28. The method is further characterized by touching one of the plurality of touch-buttons on the touch-sensitive screen 26 for navigating through the plurality of screen views 28 and for entering inputs to the control unit 24 and by pressing one of a plurality of push-buttons, generally shown at 52, on the multi-function hand controller 30 for navigating through the plurality of screens 28 and for entering inputs to the control unit 24 The plurality of screen views 28 includes the home screen view 54 shown in FIG. 3, the sensory stimulation screen view 56 shown in FIG. 4, the motor stimulation screen view 58 shown in FIG. 5, the lesion creation screen view 60 shown in FIG. 6, and the procedure summary screen view 62 shown in FIG. 7. The operator may perform sensory stimulation from the sensory stimulation screen view 56, motor stimulation from the motor stimulation screen view 58, and lesion creation from the lesion creation screen view 60.

The method proceeds by touching one of the plurality of touch-buttons on the home screen view 54 to enter input to the control unit 24 to navigate from the home screen view 54 to the sensory stimulation screen view 56. The steps are further defined by touching on the home screen view 54 either of the default settings touch-button 64, the saved procedure touch-button 66, the help touch-button 68, and the system settings touch-button 70. Touching the saved procedure touch-button 66 navigates to a saved file screen view 72 as shown in FIG. 8. The saved file screen view 72 includes file touch-buttons 74 corresponding to electrical energy specifications from previously run procedures that have been saved for reuse. The operator may select saved files from the saved file screen view 72. Touching one of the file touch-buttons 74 navigates to the sensory stimulation screen view 56 with settings for the electrical energy for that particular saved procedure.

The steps are further defined by touching either of the sensory touch button 82, the motor touch-button 84, and the lesion touch-button 86 to enter input to the control unit 24 to navigate between the plurality of screen views 28. The sensory touch-button 82, the motor touch-button 84, and the lesion touch-button 86 are presented on a menu bar 76 which is presented on each of the sensory stimulation screen view 56, the motor stimulation screen view 58, and lesion creation screen view 60. Touching the sensory touch-button 82 on the menu bar 76 navigates to the sensory stimulation screen view 56. Touching the motor touch-button 84 on the menu bar 76 navigates to the motor stimulation screen 58. Touching the lesion touch-button 86 on the menu bar 76 navigates to the lesion creation screen view 60.

The steps are further defined by pressing on the multi-function hand controller 30 either of a next push-button 88 and a back push button 90 to enter inputs to the control unit 24 to navigate between screen views. Pressing the next push-button 88 navigates to the next screen view in order. Particularly, from the sensory stimulation screen view 56, pressing the next push-button 88 will navigate to the motor stimulation screen view 58. From the motor stimulation screen view 58, pressing the next push-button 88 will navigate to the lesion creation screen view 60 and pressing the back push button 90 will navigate to the sensory stimulation screen view 56. Finally, from the lesion creation screen view 60, pressing the next push-button 88 navigates to the procedure summary screen view 62.

The steps are further defined by touching the cannula touch-button 136 to change the specification of each new cannula 36 used in the procedure.

The method is further characterized by adjusting the electrical energy to the target nerve tissue area and starting and stopping the delivery of the electrical energy to the target nerve tissue area by entering inputs to the control unit 24 with either of the multi-function hand controller 30 and the screen unit 138.

The step further includes touching the amplitude touch-button 92 to enter inputs to the control unit 24 to navigate to the amplitude adjustment screen view 98 as shown in FIG. 14. The step is further defined by touching the numbered touch-buttons 100 on the amplitude adjustment screen view 98 to enter input to the control unit 24 to change the starting amplitude specification of the stimulation energy and touching the enter touch-button 102 to set the starting amplitude specification and return to the previous screen.

The step further includes touching the frequency touch-button 94 to enter inputs to the control unit 24 to navigate to the frequency adjustment screen view 102 as shown in FIG. 9. The step is further defined by touching the numbered touch-buttons generally shown at 100 on the frequency adjustment screen view 102 to enter inputs to the control unit 24 to change the frequency setting of the stimulation energy and touching the enter touch-button 102 to set the frequency specification and return to the previous screen.

The step further includes touching the width touch-button 96 to enter inputs to the control unit 24 to navigate to the width adjustment screen view 104 as shown in FIG. 13. The step is further defined by touching the numbered touch-buttons generally shown at 100 on the width adjustment screen view 104 to enter inputs to the control unit 24 to change the width specification of the stimulation energy and touching the enter button 102 to set the width specification and return to the previous screen.

The step further includes touching the temperature limit touch-button 106 to enter inputs to the control unit 24 to navigate to a temperature limit adjustment screen view 112 as shown in FIG. 11. The step is further defined by touching the numbered touch-buttons generally shown at 100 on the temperature limit adjustment screen view 112 to enter inputs to the control unit 26 to change the temperature limit setting of the radiofrequency energy and touching the enter button 102 to set the temperature limit specification and return to the previous screen.

The step further includes touching the hold time touch-button 108 to enter inputs to the control unit 24 navigate to the hold time adjustment screen view 114 as shown in FIG. 12. The step is further defined by touching the numbered touch-buttons generally shown at 100 on the hold time adjustment screen view 114 to enter inputs to the control unit 24 to change the hold time setting of the radiofrequency energy and touching the enter button 102 to set the hold time specification and return to the previous screen.

The step is further defined by touching the start/stop touch-button 120 on the operating area 78 to enter inputs to the control unit 24 to begin or to end the delivery of the stimulation and radiofrequency output to the target nerve tissue area. The step is further defined by touching the amplitude touch-button 92 while radiofrequency is being delivered to the target nerve tissue area to enter inputs to the control unit 24 to adjust the electrical energy being delivered to the target nerve tissue area.

The step is further defined by pressing the stimulation push-button 122 on the multi-function hand controller 30 to begin the delivery of the stimulation energy to the target nerve tissue area from either of the sensory stimulation screen view 56 and the motor stimulation screen view 58. The step is further defined by pressing the increase amplitude push-button 126 during delivery of the stimulation energy to the target nerve tissue area to increase the amplitude of the stimulation energy. The step further includes pressing the decrease amplitude push-button 128 during delivery of the stimulation energy to the target nerve tissue area to decrease the amplitude of the stimulation energy. The step is further defined by pressing the lesion push-button 124 to begin the delivery of the radiofrequency energy to the target nerve tissue area from the lesion creation screen view 60. In an alternative embodiment shown in FIG. 15, the steps include pressing a fast increment adjustment button 140 to quickly adjust the amplitude of the electrical energy being delivered to the target nerve tissue area and a slow increment adjustment button 142 to slowly adjust the amplitude of the electrical energy being delivered to the target nerve tissue area.

The steps are further defined by touching a pulse mode touch-button 110 to enter inputs to the control unit 24 to navigate to the pulse mode adjustment screen view 116 as shown in FIG. 10. The step is further defined by touching the numbered touch-buttons generally shown at 100 on the pulse mode adjustment screen view 116 to enter inputs to the control unit 24 to change the pulse mode specification of the radiofrequency energy and touching the enter button 102 to set the pulse mode specification and return to the previous screen. The step is further defined by touching the on/off touch-button 118 on the pulse mode adjustment screen view 116 to turn the pulse mode on or off.

The steps are further defined by touching a summary touch-button 130 on the menu bar 76 to navigate to the procedure summary screen view 62. The procedure summary screen view 62, as shown in FIG. 7, displays a summary of the cannula selection as well as a summary of the electrical energy specifications at each of the sensory stimulation screen view 56, the motor stimulation screen view 58, and the lesion creation screen view 60.

The steps are further defined by touching a record touch-button 132 on the menu bar 76 of the procedure summary screen view 62 to save the inputs displayed on the procedure summary screen view 62. Touching the record touch-button 132 records the inputs for the electrical energy specifications as a file, which can be placed in a file, named, displayed and opened on the saved file screen view 72 as explained above and as shown in FIG. 8

The steps are further characterized by printing the hard copy of the inputs and the plurality of screen views 28 by touching one of the plurality of touch-buttons on the touch-sensitive screen 26. A print touch-button 134 is presented on the menu bar 76 and is touched to print a hard copy of the inputs for the electrical energy specifications at each of the screen views.

Obviously, many modifications and variations of the present invention are possible in light of the above teachings. The invention may be practiced otherwise than as specifically described within the scope of the appended claims, wherein that which is prior art is antecedent to the novelty set forth in the “characterized by” clause. The novelty is meant to be particularly and distinctly recited in the “characterized by” clause whereas the antecedent recitations merely set forth the old and well-known combination in which the invention resides. These antecedent recitations should be interpreted to cover any combination in which the incentive novelty exercises its utility. In addition, the reference numerals in the claims are merely for convenience and are not to be read in any way as limiting.

1. A method of operating a system (20) for performing an electrosurgical procedure using electrical energy comprising the steps of:
contacting a first electrode (22) to a target nerve tissue area of a patient for delivery of the electrical energy through the first electrode (22) to the target nerve tissue area,
manually operating a screen unit (138) to navigate through a plurality of screen views (28) and to enter inputs to a control unit (24) for controlling the delivery of electrical energy to the first electrode (22),
characterized by manually operating a hand controller (30) at the side of the patient and remote from the screen unit (138) to send control signals to the control unit (24) for controlling the delivery of electrical energy to the first electrode (22) whereby inputs to the control unit (24) may be made by either of the multi-function hand controller (30) at the patient's side and the screen unit (138) remote from the patient.

2. A method as set forth in claim 1 further characterized by navigating between the plurality of screen views (28) displayed on the screen unit (138) with either of the multi-function hand controller (30) and the screen unit (138) for entering inputs to the control unit (24) at one of the plurality of screen views (28).

3. A method as set forth in claim 1 further characterized by adjusting the electrical energy to the target nerve tissue area and starting and stopping the delivery of the electrical energy to the target nerve tissue area by entering inputs to the control unit (24) with either of the multi-function hand controller (30) and the screen unit.

4. A method as set forth in claim 1 further characterized by pressing one of a plurality of push-buttons (52) on the multi-function hand controller (30) for navigating through the plurality of screen views (28) and for entering inputs to the control unit (24).

5. A method as set forth in claim 4 further characterized by connecting the multi-function hand controller (30) to the control unit (24) by a cord (48) for establishing communication between the multi-function hand controller (30) and the control unit (24).

6. A method as set forth in claim 1 further characterized by touching a touch-sensitive screen (26) on the screen unit (138) for navigating through the plurality of screen views (28) and for entering inputs to the control unit (24).

7. A method as set forth in claim 6 further characterized by touching one of a plurality of touch-buttons on the touch-sensitive screen (26) for navigating through the plurality of screen views (28) and for entering inputs to the control unit (24).

8. A method as set forth in claim 1 further characterized by printing a hard copy of the inputs and the plurality of screen views (28) by manually operating the screen unit (138).

9. A method as set forth in claim 1 further characterized by inserting a cannula (36) into the target nerve tissue area for providing access for the first electrode (22) to the target nerve tissue area.

10. A method as set forth in claim 9 further characterized by inserting the first electrode (22) coaxially into the cannula (36) for advancing the first electrode (22) through the cannula (36) and into contact with the target nerve tissue area.

11. A method as set forth in claim 10 further characterized by inserting a stylet (38) coaxially into the cannula (36) prior to insertion of the cannula (36) into the target nerve tissue area and removing the stylet (38) from the cannula (36) after insertion of the cannula (36) into the target nerve tissue area for providing structural rigidity to the cannula (36) during insertion of the cannula (36) into the target nerve tissue area.

12. A method as set forth in claim 1 further characterized by connecting the first electrode (22) to a radiofrequency generator (34) for providing the electrical energy to the first electrode (22).

13. A method as set forth in claim 11 further characterized by connecting a second electrode (32) to the radiofrequency generator (34) and to the patient for completing an electrical circuit.

14. A method of operating a system (20) for performing an electrosurgical procedure using electrical energy comprising the steps of:
connecting a first electrode (22) and a second electrode (32) to a radiofrequency generator (34),
inserting a stylet (38) coaxially into a flexible cannula (36) for providing structural rigidity to the cannula (36),
inserting the stylet (38) and the cannula (36) into a target nerve tissue area of a patient,
removing the stylet (38) from the cannula (36) and coaxially inserting the first electrode (22) into the cannula (36) for contact with the target nerve tissue area,
contacting the second electrode (32) to the patient for completing an electrical circuit,
manually operating a screen unit (138) for navigating between the plurality of screen views (28),
manually operating a screen unit (138) for entering inputs to the control unit (24),
manually operating a screen unit (138) for beginning the delivery of the electrical energy to the target nerve tissue area,
manually operating a screen unit (138) for adjusting the inputs to the control unit (24) to adjust the electrical energy delivered to the target nerve tissue area,
manually operating a screen unit (138) for stopping the delivery of the electrical energy to the target nerve tissue area,
manually operating a screen unit (138) for printing a hard copy of the inputs and for printing the plurality of screen views (28), and
characterized by positioning a multi-function hand controller (30) at the side of the patient and remote from the screen unit (138) and operating the multi-function hand controller (30) corresponding to the screen unit (138) for navigating between the plurality of screen views (28) and for entering inputs to the control unit (24) in parallel with the screen unit (138) whereby navigation of the plurality of screen views (28) and inputs to the control unit (24) may be made by either of the multi-function hand controller (30) at the patient's side and the screen unit (138) remote from the patient.

15. A system (20) for generating electrical energy for use in an electrosurgical procedure comprising;
a first electrode (22) for contacting a target nerve tissue area of a patient and for delivering the electrical energy to the target nerve tissue area,
a control unit (24) for controlling the delivery of the electrical energy to said first electrode (22),
a screen unit (138) displaying a plurality of screen views (28) and in communication with said control unit (24) for navigating through said plurality of screen views (28) and for providing inputs to said control unit (24) for controlling the delivery of electrical energy to the first electrode (22),
characterized by a multi-function hand controller (30) in communication with said control unit (24) and remote from said screen unit (138) for providing inputs to said control unit (24) whereby an operator may position said multi-function hand controller (30) at a patient's side and enter inputs to said control unit (24) by either of said multi-function hand controller (30) and said screen unit (138).

16. A system as set forth in claim 15 wherein said multi-function hand controller (30) corresponds to said screen unit (138) for entering inputs in parallel to said control unit (24).

17. A system (20) as set forth in claim 15 wherein said multi-function hand controller (30) includes a plurality of push-buttons (52) for entering inputs to said control unit (24).

18. A system (20) as set forth in claim 17 wherein said plurality of push-buttons (52) include a next push-button (88) and a back push-button (90) and a stimulation push-button (122) and a lesion push-button (124) and an increase amplitude push-button (126) and a decrease amplitude push-button (128).

19. A system (20) as set forth in claim 17 wherein said plurality of push-buttons (52) include a next push-button (88) and a stimulation push-button (122) and a lesion push-button (124) and a fast adjustment push-button (140) and a slow adjustment push-button (142).

20. A system (20) as set forth in claim 15 wherein said multi-function hand controller (30) includes a cord (48) attaching said control unit (24) to said multi-function hand controller (30) to establish communication between said multi-function hand controller (30) and said control unit (24).

21. A system (20) as set forth in claim 15 wherein said multi-function hand controller (30) and said control unit (24) include a wireless communication system for establish wireless communication between said multi-function hand controller (30) and said control unit (24).

22. A system (20) as set forth in claim 21 wherein said wireless communication system includes an adapter in wired communication with said control unit (24) for receiving wireless signals from said multi-function hand controller (30) and for converting the wireless signals into wired signals for communication to said control unit (24).

23. A system as set forth in claim 15 wherein said screen unit includes a touch-sensitive screen (26) responsive to touching for navigating through said plurality of screen views (28) and for providing inputs to said control unit (24) for controlling the delivery of electrical energy to said first electrode (22).

24. A system (20) as set forth in claim 23 wherein said touch-sensitive screen (26) presents a plurality of touch-buttons responsive to touching for navigating through said plurality of screen views (28) and for providing inputs to said control unit (24).

25. A system (20) as set forth in claim 24 wherein said plurality of touch-buttons include a default settings touch-button (64) and a saved procedure touch-button (66) and a help touch-button (68) and a system settings touch-button (70) and a saved file touch-button (74) and a back touch-button (80) and a sensory touch-button (82) and a motor touch-button (84) and a lesion touch-button (86) and an amplitude touch-button (92) and a frequency touch-button (94) and a width touch-button (96) and numbered touch-buttons (100) an enter touch button (102) and a temperature limit touch-button (106) and a hold time touch-button (108) and a pulse mode touch-button (110) and a start/stop touch-button (120) and an on/off touch-button (118) and a summary touch-button (130) and a record touch-button (132) and a print touch-button (134) and a cannula touch-button (136).

26. A system (20) as set forth in claim 15 wherein said plurality of screen views include a home screen view (54) and a sensory stimulation screen view (56) and a motor stimulation screen view (58) and a lesion creation screen view (60) and a procedure summary screen view (62) and a saved file screen view (72) and an amplitude adjustment screen view (98) and a frequency adjustment screen view (102) and a width adjustment screen view (104) and a temperature limit adjustment screen view (112) and a hold time screen view (114) and a pulse mode adjustment screen view (116).

27. A system (20) as set forth in claim 15 including a cannula (36) for providing access for said first electrode (22) to the target nerve tissue area.

28. A system (20) as set forth in claim 27 including a stylet (38) coaxially insertable into and removable from said cannula (36) for providing structural rigidity for insertion of said cannula (36) into the target nerve tissue area and for removal of said stylet (38) after insertion of said cannula (36) into the target nerve tissue area.

29. A system (20) as set forth in claim 15 including a radiofrequency generator (34) in communication with said first electrode (22) and controlled by said control unit (24) for providing the electrical energy to said first electrode (22).

30. A system (20) as set forth in claim 15 further including a second electrode (32) in communication with said radiofrequency generator (34) and in contact with the patient for completing an electrical circuit.

31. A system as set forth in claim 15 including a printer (50) in communication with said control unit (24) for printing a hard copy of the inputs provided to said control unit (24) and for printing said plurality of screen views (28).

32. A system (20) for generating electrical energy for use in an electrosurgical procedure comprising;
a flexible cannula (36),
a stylet (38) coaxially insertable into and removable from said cannula (36) for providing structural rigidity for insertion of said cannula (36) into a target nerve tissue area of a patient and for removal of said stylet (38) after insertion of said cannula (36) into the target nerve tissue area,
a radiofrequency generator (34) for providing the electrical energy,
a first electrode (22) in communication with said radiofrequency generator (34) for insertion into said cannula (36) after removal of said stylet (38) to contact the target nerve tissue area and for delivering the electrical energy to the target nerve tissue area,
a second electrode (32) for contacting a patient for completing an electrical circuit,
a control unit (24) in communication with said radiofrequency generator (34) for controlling said radiofrequency generator (34),
a screen unit (138) in communication with said control unit (24) to display a plurality of screen views (28) for providing inputs to said control unit (24),
a printer (50) in communication with said control unit (24) for printing a hard copy of the inputs provided to said control unit (24) and for printing said plurality of screen views (28), and
characterized by a multi-function hand controller (30) being remote from said screen unit (138) and in communication with said control unit (24) and corresponding to said screen unit (138) for navigating between each of said plurality of screen views (28) in parallel with said screen unit (138) and for providing inputs to said control unit (24) in parallel with said screen unit (138) whereby an operator may position said multi-function hand controller (30) at a patient's side and navigate between each of said plurality of screen views (28) and enter inputs to said control unit (24) by either of said multi-function hand controller (30) and said screen unit (138).
